(19) United States
(12) Patent Application Publication
(10) Pub. No.: US 2017/0253890 A1
(43) Pub. Date: Sep. 7, 2017

(54) METHOD FOR PREPARING BACTERIAL AGENT FOR REMOVING AMMONIA

(71) Applicant: Nanjing University, Nanjing (CN)

(72) Inventors: Jinju GENG, Nanjing (CN); Hongqiang REN, Nanjing (CN); Ke XU, Nanjing (CN); Lili DING, Nanjing (CN)

(21) Appl. No.: 15/373,397

(22) Filed: Dec. 8, 2016

Related U.S. Application Data
(63) Continuation-in-part of application No. 15/361,064, filed on Nov. 24, 2016.

(30) Foreign Application Priority Data
Oct. 8, 2015 (CN) 201510640081.6

(51) Int. Cl. C12P 1/04; C02F 3/34; C02F 3/30

(52) U.S. Cl. CPC C12P 1/04; C02F 3/302; C02F 3/341; C02F 2101/16

(57) ABSTRACT

A method for preparing a bacterial agent for removing ammonia-nitrogen, including: 1) activating a mixed microbial preparation including heterotrophic nitrification bacteria and aerobic denitrification bacteria; 2) inoculating the microbial preparation including heterotrophic nitrification bacteria and the aerobic denitrification bacteria to a membrane region including a double-layered filler; 3) introducing a culture solution of the microbial preparation to the membrane region from one side of the membrane region at a certain pressure and a certain flow rate to cultivate bacteria and discharging metabolites produced by the microbial preparation from the other side of the membrane region; 4) centrifuging a bacterial liquid obtained from cultivation to yield a concentrated bacterial suspension; and 5) adding a protecting agent to the concentrated bacterial suspension, uniformly dry spraying a resulting mixture to a sterilized carrier to yield a bacterial agent, and sealing the bacterial agent for storage.

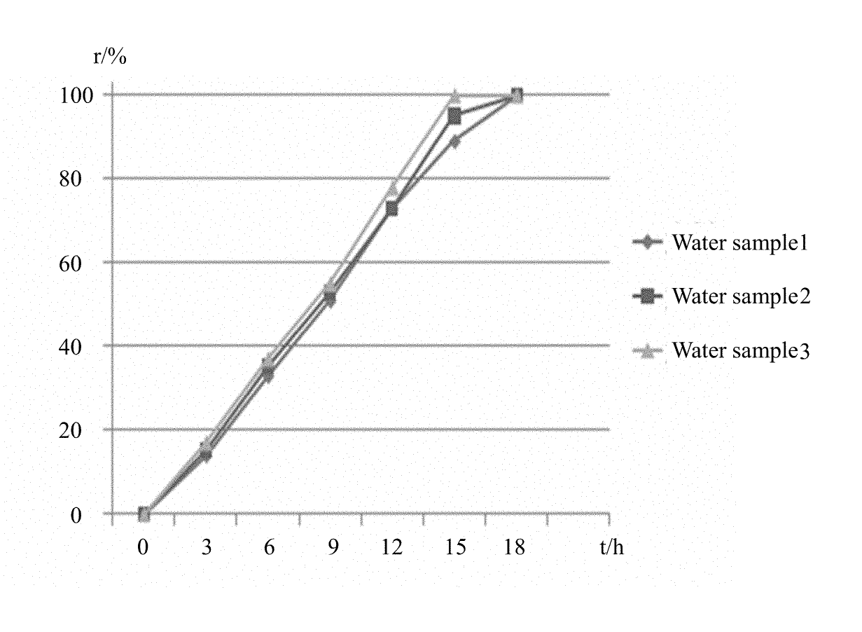

### CROSS-REFERENCE TO RELATED APPLICATIONS

Pursuant to 35 U.S.C. §119 and the Paris Convention Treaty, this application claims the benefit of Chinese Patent Application No. 201510640081.6 filed Oct. 8, 2015, the contents of which are incorporated herein by reference.

### BACKGROUND OF THE INVENTION

Field of the Invention

The invention relates to a method for preparing a bacterial agent for removing ammonia in water.

Description of the Related Art

Conventional biological processes of nitrogen removal include nitrification and denitrification, which cannot happen synchronously because the nitrification needs oxygen and the denitrification excludes oxygen. A two-stage biological nitrogen removal process is costly, energy-consuming, and complex for operation.

In addition, metabolites produced by bacteria during the cultivation of a mixed bacterial culture comprising nitrifying bacteria and denitrifying bacteria cannot be discharged timely, which inhibits mass reproduction of the mixed culture.

### SUMMARY OF THE INVENTION

In view of the above-described problems, it is one objective of the invention to provide a method for preparing a bacterial agent comprising nitrifying bacteria and aerobic denitrifying bacteria.

Using the method, the nitrifying bacteria and the aerobic denitrifying bacteria can be synchronously cultured at an optimal growth status, and then are prepared to yield a mixed microbial agent for ammonia-nitrogen removal.

To achieve the above objective, in accordance with one embodiment of the invention, there is provided a method for preparing a mixed nitrifying bacterial and denitrifying bacterial agent.

The method comprises:

1) activating a mixed microbial preparation comprising heterotrophic nitrification bacteria and aerobic denitrification bacteria;
2) inoculating the microbial preparation comprising the nitrification bacteria and aerobic denitrification bacteria to a membrane region comprising a double-layered filler;
3) introducing a culture solution of the microbial preparation to the membrane region from one side of the membrane region at a certain pressure and a certain flow rate to cultivate bacteria and discharging metabolites produced by the microbial preparation from the other side of the membrane region;
4) centrifuging a bacterial liquid obtained from cultivation to yield a concentrated bacterial suspension; and
5) adding a protecting agent to the concentrated bacterial suspension, uniformly dry spraying a resulting mixture to a sterilized carrier to yield a denitrifying bacterial agent, and sealing the denitrifying bacterial agent for storage.

The double-layered filler allows the culture solution and the metabolite produced by the microbial preparation to pass through and prevent the microbial preparation from penetrating. The culture solution comprises: between 20 and 26 parts by weight of CH₃COONa, between 22 and 25 parts by weight of Na₂CO₃, between 12 and 15 parts by weight of NH₄Cl, between 0.05 and 1.0 parts by weight of FeSO₄, between 3 and 5 parts by weight of MgSO₄.7H₂O, and between 380 and 500 parts by weight of a phosphate buffer. The phosphate buffer is prepared by K₂HPO₃ and KH₂PO₄ according to a weight ratio of 3:1.

In a class of this embodiment, the microbial preparation is a mixture of heterotrophic nitrifying bacteria and the aerobic denitrifying bacteria.

In a class of this embodiment, the certain pressure applied on the culture solution is between 0.01 and 0.05 megapascal. The certain flow rate of the culture solution is equivalent to that the culture solution flows into the membrane region in a unit time accounts for between 0.002 and 0.003 fold of a total volume of the culture solution within the membrane region.

In a class of this embodiment, the activation of the microbial preparation comprising the aerobic denitrification bacteria comprises: inoculating 10 parts by weight of the mixed microbial preparation comprising the heterotrophic nitrifying bacteria and the aerobic denitrifying bacteria into an 800 mL conical flask containing 150 mL of a sterilized water, shaking the microbial preparation comprising heterotrophic nitrifying bacteria and the aerobic denitrification bacteria at a rotational speed of 220 rpm for between 2.5 and 3 hrs, and discarding an original carrier.

In a class of this embodiment, a temperature of the cultivation process is controlled at between 29 and 32° C.

In a class of this embodiment, a pH value of the culture solution is controlled at between 7.0 and 8.0. Too high or too low of the pH value is not beneficial for the growth of the bacteria.

In a class of this embodiment, a concentration of a dissolved oxygen in the culture solution is between 0.6 and 0.8 mg/L. When the concentration of the dissolved oxygen is lower than 0.6 mg/L, the growth of the nitrifying bacteria becomes slow, and too high of the concentration of the dissolved oxygen results in surplus.

In a class of this embodiment, the centrifuging is conducted at a rotational speed of between 2000 and 2300 rpm for 15 min.

In a class of this embodiment, the protecting agent is a mixture of 0.8 percent by weight of glycine and 2.0 percent by weight of glycerin according to a ratio of glycine to glycerin of between 1:2 and 1:3; and an addition of the protecting agent is between 2.2 and 3.6 folds of the concentrated bacterial suspension.

In a class of this embodiment, the carrier is calcium alginate, a diatomite, or a rice bran.

Advantages of the method for preparing the mixed bacterial agent for removing ammonia-nitrogen according to embodiments of the invention are summarized as follows: the heterotrophic nitrifying bacteria and the aerobic denitrifying bacteria are inoculated in the membrane region comprising a double-layered filler for mixed cultivation. The culture solution is introduced to the membrane region from one side of the membrane region at a certain pressure and a certain flow rate to supply the growth of the bacteria and to carrier away the bacterial metabolite from the other side of the membrane region. In the meanwhile, the heterotrophic nitrifying bacteria supply aerobic denitrification bacteria with nitrate nitrogen necessitated for the growth of the aerobic denitrification bacteria, and the aerobic denitrification bacteria remove the growing obstacles for the heterotrophic nitrifying bacteria, so that both the nitrifying bacteria and the denitrifying bacteria are in the optimal growing status, realizing concentrated cultivation of the bacteria and the production of the bacterial agent that is easily preserved after amplifying cultivation. After long term preservation, the mixed bacterial agent for removing ammonia-nitrogen still works effectively in degrading ammonia-nitrogen and possesses good reproduction and activation properties.

### BRIEF DESCRIPTION OF THE DRAWINGS

The invention is described hereinbelow with reference to accompanying drawings, in which the sole figure is a chart showing removal effect of ammonia-nitrogen of a denitrifying bacterial agent in accordance with one embodiment of the invention.

### DETAILED DESCRIPTION OF THE EMBODIMENTS

For further illustrating the invention, experiments detailing a method for preparing a mixed nitrifying bacterial and denitrifying bacterial agent are described below. It should be noted that the following examples are intended to describe and not to limit the invention.

### Example 1

A method for preparing a bacterial agent for removing ammonia-nitrogen was conducted as follows: 10 parts by weight of a mixed bacterial culture of nitrifying bacteria and aerobic denitrifying bacteria was inoculated into an 800 mL of a conic flask containing 150 mL of a sterilized water and shaken at a rotational speed of 220 rpm for activation for 2.5 hrs. Thereafter, an original carrier was discarded and the mixed bacterial culture was inoculated into a membrane region comprising a double-layered filler. The filler was a hollow fiber membrane in a planar shape, and a thickness of the filler was 0.1 mm. The double-layered filler allowed a culture solution and bacterial metabolites to pass through and prevented the bacteria from passing through. The culture solution flowed into the membrane region from one side of the membrane region at a pressure of 0.01 megapascal at a certain flow rate. The flow rate was equivalent to that the culture solution entering the membrane region in a unit time was 0.002 fold of a total volume of the culture solution in the membrane region. The culture solution was adapted to supply the bacteria with nutrition for growth and to carry the bacterial metabolites out of the membrane region from the other side of the membrane region. Concentrated cultivation of the bacteria was performed at a temperature of 29° C. and a pH value of 7.0. Too high or too low of the pH value was not beneficial for the growth of the bacteria. The concentration of the dissolved oxygen in the culture solution was 0.6 mg/L. When the concentration of the dissolved oxygen is lower than 0.6 mg/L, the growth of the nitrifying bacteria was slow, and too high the concentration of the dissolved oxygen resulted in surplus. The bacteria solution after cultivation was then centrifuged at the rotational speed of 2000 rpm for 15 min, and the bacteria were separated to prepare a concentrated bacterial suspension. 0.8 percent by weight of glycine and 2.0 percent by weight of glycerin were mixed according to a weight ratio of glycine to glycerin of 1:2 to prepare a protecting agent and the protecting agent was then added to the concentrated bacterial suspension, in which an addition of the protecting agent was 2.2 folds of the concentrated bacterial suspension. The resulting mixture was uniformly loaded on sterilized calcium alginate as a carrier by dry spraying to yield the denitrifying bacterial agent, in which, a water content of the carrier was controlled equal to or less than 12 percent by weight. The yielded bacterial agent was sealed for storage. The culture solution comprised: 20 parts by weight of CH₃COONa, 22 parts by weight of Na₂CO₃, 12 parts by weight of NH₄Cl, 0.05 parts by weight of FeSO₄, 3 parts by weight of MgSO₄.7H₂O, and 380 parts by weight of a phosphate buffer. The phosphate buffer was prepared by K₂HPO₃ and KH₂PO₄ in a weight ratio of 3:1.

### Example 2

A method for preparing a bacterial agent for removing ammonia-nitrogen was conducted as follows: 10 parts by weight of a mixed bacterial culture of nitrifying bacteria and aerobic denitrifying bacteria was inoculated into an 800 mL of a conic flask containing 150 mL of a sterilized water and shaken at a rotational speed of 220 rpm for activation for 2.75 hrs. Thereafter, an original carrier was discarded and the mixed bacterial culture was inoculated into a membrane region comprising a double-layered filler. The filler was a hollow fiber membrane in a tubular shape, and a thickness of the filler was 0.55 mm. The double-layered filler allowed a culture solution and bacterial metabolites to pass through and prevented the bacteria from passing through. The culture solution flowed into the membrane region from one side of the membrane region at a pressure of 0.03 megapascal at a certain flow rate. The flow rate was equivalent to that the culture solution entering the membrane region in a unit time was 0.0025 fold of a total volume of the culture solution in the membrane region. The culture solution was adapted to supply the bacteria with nutrition for growth and to carry the bacterial metabolites out of the membrane region from the other side of the membrane region. Concentrated cultivation of the bacteria was performed at a temperature of 30.5° C. and a pH value of 7.5. Too high or too low of the pH value was not beneficial for the growth of the bacteria. The concentration of the dissolved oxygen in the culture solution was 0.7 mg/L. When the concentration of the dissolved oxygen is lower than 0.6 mg/L, the growth of the nitrifying bacteria was slow, and too high the concentration of the dissolved oxygen resulted in surplus. The bacteria solution after cultivation was then centrifuged at the rotational speed of 2150 rpm for 15 min, and the bacteria were separated to prepare a concentrated bacterial suspension. 0.8 percent by weight of glycine and 2.0 percent by weight of glycerin were mixed according to a weight ratio of glycine to glycerin of 1:2.5 to prepare a protecting agent and the protecting agent was then added to the concentrated bacterial suspension, in which an addition of the protecting agent was 2.9 folds of the concentrated bacterial suspension. The resulting mixture was uniformly loaded on a sterilized diatomite as a carrier by dry spraying to yield the denitrifying bacterial agent, in which, a water content of the carrier was controlled less than or equal to 13.5 percent by weight. The yielded denitrifying bacterial agent was sealed for storage. The culture solution comprised: 23 parts by weight of CH₃COONa, 23.5 parts by weight of Na₂CO₃, 13.5 parts by weight of NH₄Cl, 0.525 part by weight of FeSO₄, 4parts by weight of MgSO₄.7H₂O, and 420 parts by weight of a phosphate buffer. The phosphate buffer was prepared by K₂HPO₃ and KH₂PO₄ in a weight ratio of 3:1.

### Example 3

A method for preparing a bacterial agent for removing ammonia-nitrogen was conducted as follows: 10 parts by weight of a mixed bacterial culture of nitrifying bacteria and aerobic denitrifying bacteria was inoculated into an 800 mL of a conic flask containing 150 mL of a sterilized water and shaken at a rotational speed of 220 rpm for activation for 3 hrs. Thereafter, an original carrier was discarded and the mixed bacterial culture was inoculated into a membrane region comprising a double-layered filler. The filler was a capillary membrane in a tubular shape and a thickness of the filler was 1.0 mm. The double-layered filler allowed a culture solution and bacterial metabolites to pass through and prevented the bacteria from passing through. The culture solution flowed into the membrane region from one side of the membrane region at a pressure of 0.05 megapascal at a certain flow rate. The flow rate was equivalent to that the culture solution entering the membrane region in a unit time was 0.003 fold of a total volume of the culture solution in the membrane region. The culture solution was adapted to supply the bacteria with nutrition for growth and to carry the bacterial metabolites out of the membrane region from the other side of the membrane region. Concentrated cultivation of the bacteria was performed at a temperature of 32° C. and a pH value of 8.0. Too high or too low of the pH value was not beneficial for the growth of the bacteria. The concentration of the dissolved oxygen in the culture solution was 0.8 mg/L. When the concentration of the dissolved oxygen is lower than 0.6 mg/L, the growth of the nitrifying bacteria was slow, and too high the concentration of the dissolved oxygen resulted in surplus. The bacteria solution after cultivation was then centrifuged at the rotational speed of 2300 rpm for 15 min, and the bacteria were separated to prepare a concentrated bacterial suspension. 0.8 percent by weight of glycine and 2.0 percent by weight of glycerin were mixed according to a weight ratio of glycine to glycerin of 1:3 to prepare a protecting agent and the protecting agent was then added to the concentrated bacterial suspension, in which an addition of the protecting agent was 3.6 folds of the concentrated bacterial suspension. A resulting mixture was uniformly loaded on a sterilized rice bran as a carrier by dry spraying to yield the denitrifying bacterial agent, in which, a water content of the carrier was controlled within 15 percent by weight. The yielded denitrifying bacterial agent was sealed for storage. The culture solution comprised: 26 parts by weight of CH₃COONa, 25parts by weight of Na₂CO₃, 15 parts by weight of NH₄Cl, 1.0 part by weight of FeSO₄, 5 parts by weight of MgSO₄.7H₂O, and 500 parts by weight of a phosphate buffer. The phosphate buffer was prepared by K₂HPO₃ and KH₂PO₄ in a weight ratio of 3:1.

The heterotrophic nitrifying bacteria and the aerobic denitrifying bacteria are placed in the membrane region formed by the double-layered filler for mixed cultivation. The culture solution is introduced to the membrane region from one side of the membrane region at a certain pressure and a certain flow rate to supply the growth of the bacteria and to carrier away the bacterial metabolite from the other side of the membrane region. In the meanwhile, the heterotrophic nitrifying bacteria supply aerobic denitrifying bacteria with nitrate nitrogen necessitated by the growth of the aerobic denitrifying bacteria, and the aerobic denitrifying bacteria remove the growing obstacles for the heterotrophic nitrifying bacteria, so that both the nitrifying bacteria and the denitrifying bacteria are in best growing status, realizing concentrated cultivation of the bacteria and the production of the bacterial agent that is easily preserved after amplifying cultivation. After long term preservation, the mixed nitrifying bacterial and denitrifying bacterial agent still works effectively in degrading ammonia-nitrogen and possesses good reproduction and activation qualities. The bacteria possess good growth potential and are effective to remove ammonia-nitrogen.

Experimental Verification

The mixed bacterial agents prepared by the methods of Examples 1-3 were taken out after one-year preservation and added to three groups of the same water sample to be treated, in which a concentration of the ammonia-nitrogen was tested to be 0.24 g nitrogen per mL, for demonstrating the removal effect of the ammonia-nitrogen. Data were collected and a graph shown in the sole figure was charted, which indicates that the mixed bacterial agents have good effects in treating the water sample, and the removal rate of the ammonia-nitrogen reaches 100 percent by weight after 18 hrs.

Unless otherwise indicated, the numerical ranges involved in the invention include the end values. While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A method for preparing a bacterial agent, the method comprising:
1) activating a microbial preparation comprising heterotrophic nitrification bacteria and aerobic denitrification bacteria;
2) inoculating the microbial preparation comprising the heterotrophic nitrification bacteria and aerobic denitrification bacteria to a membrane region comprising a double-layered filler;
3) introducing a culture solution of the microbial preparation to the membrane region from one side of the membrane region at a certain pressure and a certain flow rate to cultivate and concentrate the microbial preparation and discharging metabolites produced by the microbial preparation from the other side of the membrane region;
4) centrifuging a bacterial liquid obtained from cultivation in 2) to yield a concentrated bacterial suspension; and
5) adding a protecting agent to the concentrated bacterial suspension, uniformly dry spraying a resulting mixture to a sterilized carrier to yield a bacterial agent, and sealing the bacterial agent for storage;
wherein
the double-layered filler allows the culture solution and the metabolite produced by the microbial preparation to pass through and prevent the microbial preparation from penetrating; and
the culture solution comprises: between 20 and 26 parts by weight of CH3COONa, between 22 and 25 parts by weight of Na₂CO₃, between 12 and 15 parts by weight of NH4Cl, between 0.05 and 1.0 parts by weight of FeSO4, between 3 and 5 parts by weight of MgSO4.7H2O, and between 380 and 500 parts by weight of a phosphate buffer.

2. The method of claim 1, wherein the microbial preparation comprising the heterotrophic nitrification bacteria and aerobic denitrification bacteria is a mixture of nitrifying bacteria and aerobic denitrifying bacteria.

3. The method of claim 1, wherein the certain pressure applied on the culture solution is between 0.01 and 0.05 megapascal; the certain flow rate of the culture solution is equivalent to that the culture solution flows into the membrane region in a unit time accounts for between 0.002 and 0.003 fold of a total volume of the culture solution within the membrane region.

4. The method of claim 1, wherein the activation of the microbial preparation comprising the heterotrophic nitrification bacteria and aerobic denitrification bacteria comprises: inoculating 10 parts by weight of the microbial preparation comprising the heterotrophic nitrification bacteria and aerobic denitrification bacteria into an 800 mL conical flask containing 150 mL of a sterilized water, shaking the microbial preparation comprising the heterotrophic nitrification bacteria and aerobic denitrification bacteria at a rotational speed of 220 rpm for between 2.5 and 3 hrs, and discarding an original carrier.

5. The method of claim 1, wherein a temperature of the cultivation process is controlled at between 29 and 32° C.

6. The method of claim 1, wherein a pH value of the culture solution is controlled at between 7.0 and 8.0.

7. The method of claim 1, wherein a concentration of a dissolved oxygen in the culture solution is between 0.6 and 0.8 mg/L.

8. The method of claim 1, wherein the centrifuging is conducted at a rotational speed of between 2000 and 2300 rpm for 15 min.

9. The method of claim 1, wherein the protecting agent is a mixture of glycine and glycerin according to a ratio of the glycine to the glycerin of between 1:2 and 1:3; and an addition of the protecting agent is between 2.2 and 3.6 folds of the concentrated bacterial suspension.

10. The method of claim 1, wherein the carrier is calcium alginate, a diatomite, or a rice bran.
